(19) United States
(12) Patent Application Publication
(10) Pub. No.: US 2011/0189241 A1
(43) Pub. Date: Aug. 4, 2011

(54) Process For Controlling The Particle Size of A [3-(Trifluoromethyl)Phenyl]-1-Aminopropane Derivative

(75) Inventor: Bernardino Mangion, Santa Lucia (MT)

(73) Assignee: Medichem, S.A., Sant Joan Despi, Barcelona (ES)

(21) Appl. No.: 12/990,895

(22) PCT Filed: May 4, 2009
(86) PCT No.: PCT/US09/42653

§ 371 Date: Jan. 24, 2011

Related U.S. Application Data
(60) Provisional application No. 61/095,880, filed on Sep. 10, 2008.
(60) Provisional application No. 61/050,527, filed on May 5, 2008.

(51) Int. Cl. A61K 9/14; C07C 209/82; A61K 31/137; A61P 5/18; A61P 3/00; B32B 5/16

(52) U.S. Cl. 424/400; 564/387; 514/655; 428/402

(57) ABSTRACT

The invention relates to a process for tightly controlling the particle size of cinacalcet hydrochloride, i.e. a process for preparing large or small crystals of cinacalcet hydrochloride, which yields cinacalcet hydrochloride in a narrow, reproducible and consistent distribution of particles, which hence does not require to reprocess, rework or destroy material of undesired size, which is efficient and cost-effective, and which is suitable for industrial implementation.

### CROSS REFERENCE TO RELATED APPLICATIONS

This application claims priority to U.S. Provisional Application Nos. 61/050,527 and 61/095,880, filed May 5, 2008 and Sep. 10, 2008 respectively, which applications are expressly incorporated herein by reference in their entirety.

### BACKGROUND OF THE INVENTION

1. Field of the Invention

The present invention relates to a process for controlling the particle size of cinacalcet hydrochloride, i.e. a process for preparing large or small crystals of cinacalcet hydrochloride.

2. Relevant Background

Cinacalcet hydrochloride is a commercially marketed pharmaceutically active substance known to be useful for the treatment of hyperparathyroidism and the preservation of bone density in patients with kidney failure or hypercalcemia due to cancer. Cinacalcet hydrochloride is the generic international denomination for N-[1-(R)-(−) (R)-(−)-(1-naphthyl)ethyl]-3-[3-(trifluoromethyl)phenyl]-1-aminopropane hydrochloride, which has the formula (I) given below:

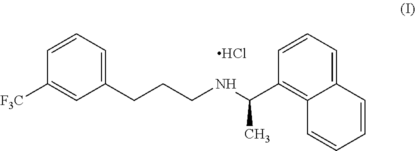

Cinacalcet hydrochloride is an oral calcimimetic drug. In the United States, it is marketed under the name Sensipar® and, in Europe, it is marketed under the name Mimpara® and Parareg®. It has been approved for the treatment of secondary hyperparathyroidism in patients with chronic kidney disease on dialysis and for the treatment of hypercalcemia in patients with parathyroid carcinoma.

U.S. Pat. No. 7,247,751 discloses that the crystalline cinacalcet hydrochloride currently marketed as Sensipar® is characterized as crystalline Form I, and encompasses processes for its preparation. Among other processes, U.S. Pat. No. 7,247,751 generally describes a process for preparing cinacalcet hydrochloride by means of (i) dissolving cinacalcet hydrochloride in a C3-6 ketone, C1-C5 straight or branched alcohol, and (ii) precipitating the same with an anti-solvent.

The Scientific Discussion for the European Public Assessment Report published by the EMEA mentions that cinacalcet hydrochloride has a very low aqueous solubility. In this regard, the EMEA mentions the importance that the particle size and physical form of cinacalcet hydrochloride have on the dissolution and hence on the bioavailability of the active substance. Furthermore, EMEA states that the particle size of cinacalcet hydrochloride needs to be tightly controlled to ensure the clinical safety and efficacy of the medicinal product.

The relevance of the particle size of cinacalcet hydrochloride has been only tackled in U.S. Patent Application Publication No. US 2005/0147669 A1. This reference describes, among other compositions, a pharmaceutical composition comprising cinacalcet hydrochloride as active pharmaceutical ingredient, wherein the composition has a controlled dissolution profile. To this purpose, it is described that the cinacalcet hydrochloride used in the composition typically has a mass median diameter (i.e. D₅₀) less than or equal to about 50 μm. Additionally, it also states that the size of the particles is controlled during the production of the active pharmaceutical ingredient (e.g. cinacalcet hydrochloride), for example, by use of a milling step, or a controlled crystallization process. More precisely, this reference discloses that the active pharmaceutical ingredient can be milled using a stainless steel hammer mill with 5 mm screen and 12 hammers forward at a mill speed of 8100±100 rpm, with the feed speed is set a 90±10 rpm. However, US 2005/0147669 A1 only provides examples focused on the control of the granules of a number of pharmaceutical formulations containing cinacalcet hydrochloride.

In view of the aforesaid, no examples aimed at control of the particle size of active pharmaceutical ingredient cinacalcet hydrochloride are provided in the literature. In addition, although suggested in US 2005/0147669 A1, no crystallization process to control the particle size of cinacalcet hydrochloride has been reported in the prior art so far. Further, the only technique suggested in US 2005/0147669 A1 to control the size of the particles (i.e. milling cinacalcet hydrochloride) presents a number of disadvantages. Namely, the method of milling cinacalcet hydrochloride regardless of the process used to prepare the same, might allow for the production of cinacalcet hydrochloride with a broad, irreproducible and inconsistent distribution of particle size which might require reprocessing, reworking or destroying those particles outside of the required distribution. More precisely, a starting feedstock of cinacalcet hydrochloride that has a wide distribution of particle sizes will yield a reduced material still with a wide particle size distribution because the same amount of energy of the hammer has been imparted to all of the particles regardless of their size. Additionally, the method of milling described in this reference can not be regarded as a method to control the particle size of cinacalcet hydrochloride, since it is only limited to a process for the reduction of the size of said particles. Thus, in view of the above, this method can be time consuming, costly, and not suitable for industrial implementation if reprocessing, reworking, or destruction of the material with undesired size is necessary.

Therefore there is the need to provide an improved process for tightly controlling the particle size of cinacalcet hydrochloride which might yield cinacalcet hydrochloride with a narrow, reproducible and consistent distribution of particles, which hence avoids the need to reprocess, rework or destroy material of undesired size, and which therefore might be more efficient, economic, and suitable for industrial implementation.

### SUMMARY OF THE INVENTION

The present invention relates to a process for controlling the particle size of cinacalcet hydrochloride, i.e. a process for preparing large or small crystals of cinacalcet hydrochloride.

The invention relates to a process for tightly controlling the particle size of cinacalcet hydrochloride, i.e. a process for preparing large or small crystals of cinacalcet hydrochloride, which yields cinacalcet hydrochloride in a narrow, reproducible and consistent distribution of particles, which hence does not require to reprocess, rework or destroy material of undesired size, which is efficient and cost-effective, and which is suitable for industrial implementation.

As used herein, “small crystals” of cinacalcet hydrochloride is intended to encompass those crystals which have a mass median diameter (i.e. D₅₀) less than or equal to about 50 μm. Likewise, “large crystals” of cinacalcet hydrochloride is intended to encompass those crystals which have a D₅₀ higher than about 50 μm.

It has been found that when the crystallization of cinacalcet hydrochloride is carried out under ordinary cooling conditions, i.e. at a mean cooling rate higher than about 22° C./h, the cinacalcet hydrochloride obtained shows small crystals with a distribution of D_{[v, 0.5]} of 20.7 μm to 49.8 μm, and a distribution of D_{[v, 0.9]} of 95.4 μm to 387.9 μm.

It has also been found that if the cinacalcet hydrochloride is crystallized under controlled cooling conditions, the obtained cinacalcet hydrochloride shows large crystals with a narrow, reproducible and consistent particle size distribution. Additionally, the said crystallization under controlled cooling conditions and results are reproducible at higher scales. Further, said large crystals with narrow particle size distribution obtained can be easily isolated by filtration, and can be used to prepare small crystals with narrow particle size distribution. So, the process of the invention is useful to control the particle size of cinacalcet hydrochloride.

The various embodiments of the invention having thus been generally described, several examples will hereafter be discussed to illustrate the inventive aspects more fully.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein.

In particular, a first aspect of the present invention relates to a process for preparing large crystals of cinacalcet hydrochloride, wherein said large crystals of cinacalcet hydrochloride show a narrow particle size distribution, said process comprising crystallizing cinacalcet hydrochloride under controlled cooling conditions. Namely, as used herein, the phrase “controlled cooling conditions” comprise a controlled mean cooling rate lower than about 22° C./h. As used herein, a “hot solution” is intended to encompass a solution having a temperature of not less than about 75° C.

The cooling rate value is calculated as the variation of Celsius degrees temperature per hour, and it is expressed as the absolute value.

In a further aspect, the process for preparing large crystals of cinacalcet hydrochloride of the invention comprises the steps of (i) providing a hot solution of cinacalcet hydrochloride and a solvent comprising an organic solvent, wherein said hot solution has a temperature not less than about 75° C., (ii) allowing for the presence of crystals, at a temperature not less than about 75° C., (iii) cooling at a controlled mean cooling rate lower than about 22° C./h until the temperature is reduced at least 10° C., to obtain a suspension, (iv) allowing the suspension to achieve at least room temperature, (v) isolating large crystals of cinacalcet hydrochloride with a narrow particle size distribution from said suspension, and (vi) optionally, drying said cinacalcet hydrochloride.

The controlled median cooling rate is lower than about 22° C./h of step (iii) of the process of the invention, preferably is lower than about 10° C./h, and more preferably is equal or lower than about 1° C./h. Surprisingly, it has been found that the use of a controlled mean cooling rate lower than about 22° C./h is a key step for obtaining large crystals of cinacalcet hydrochloride with a narrow particle size distribution.

The solvent comprising an organic solvent preferably is at least one organic solvent or mixtures of at least one organic solvent and water, and more preferably is at least one organic solvent.

The at least one organic solvent preferably is at least one of an alcohol solvent, a ketonic solvent, an ester solvent, an ether solvent, a polar aprotic solvent, or mixtures thereof, more preferably is an ester solvent, and even more preferably is isobutyl acetate.

Suitable alcoholic solvents include, but are not limited to, C1 to C4 straight or branched chain alcohol solvents or mixtures thereof, and in particular are methanol, ethanol, n-propanol, 2-propanol, 2-butanol, n-butanol, or mixtures thereof.

Suitable ketonic solvents include, but are not limited to, acetone, methyl ethyl ketone, methyl isopropyl ketone, or mixtures thereof, and in particular are acetone, methyl ethyl ketone, or mixtures thereof.

Suitable ester solvents include, but are not limited to, ethyl acetate, propyl acetate, isobutyl acetate, isopropyl acetate, or mixtures thereof, and more particularly is isobutyl acetate.

Suitable ether solvents include, but are not limited to, diethylether, methyl tert-butyl ether, cyclic ethers, or mixtures thereof, and in particular are tetrahydrofuran, 1,4-dioxane, 2-methyltetrahydrofuran, 1,3-dioxolane, or mixtures thereof.

Suitable polar aprotic solvents include, but are not limited to, N,N-dimethylformamide, dimethylsulfoxide, dimethylacetamide, acetonitrile, or mixtures thereof.

The hot solution of cinacalcet hydrochloride and a solvent comprising an organic solvent having a temperature not less than about 75° C. of step (i), preferably has a temperature within the range between about 115-85° C.

In an embodiment of the invention, the allowing for the presence of crystals, at a temperature not less than about 75° C. of step (ii) comprises spontaneous formation of crystals of cinacalcet hydrochloride.

In an alternative embodiment of the invention, the allowing for the presence of crystals, at a temperature not less than about 75° C. of step (ii) comprises seeding the hot solution with seeds of cinacalcet hydrochloride.

Preferably, the seeding of the hot solution with cinacalcet hydrochloride comprises seeding with between about 0.05-10% w/w of cinacalcet hydrochloride.

In some embodiments, the seeds of cinacalcet hydrochloride have a D₅₀ equal or less than about 50 μm, and more particularly have a D₅₀ between about 12-50 μm. But seeds of cinacalcet hydrochloride having a D₅₀ higher than 50 μm may be also used.

The cooling at a controlled mean cooling rate lower than about 22° C./h until the temperature is reduced at least 10° C. of step (iii) of the process above, preferably comprises cooling at a controlled mean cooling rate lower than about 22° C./h until a temperature within the range of about 85-65° C.

The allowing the suspension to achieve at least room temperature of step (iv) of the process above, preferably comprises cooling the suspension until a temperature within the range of about 5-0° C.

The isolating large crystals of cinacalcet hydrochloride with a narrow particle size distribution from said suspension of step (v) of the process above, preferably comprises filtering the suspension.

The cinacalcet hydrochloride used in the process for controlling the particle size of the invention can be either cinacalcet hydrochloride obtained by a known method.

In another aspect, the large crystals of cinacalcet hydrochloride with a narrow particle size distribution obtained by the process of the invention show a mass median diameter (i.e. D₅₀) higher than 50 μm. In some embodiments, the large crystals of cinacalcet hydrochloride obtained by the process of the invention have a distribution of D_{[v, 0.5]} of 57.7 μm to 99.8 μm.

In an additional aspect, the large crystals of cinacalcet hydrochloride with a narrow particle size distribution obtained by the process of the invention have a D₉₀ lower than about 550 μm. In some embodiments, the large crystals of cinacalcet hydrochloride obtained by the process of the invention have a distribution of D_{[v, 0.9]} of 392.3 μm to 518.3 μm.

It is noted that the notation D_{X} (or D_{[v, 0.X]}) means that X % of the particles have a diameter less than a specified diameter D. Thus a D₉₀ (or D_{[v, 0.9]}) of 550 μm means that 90% of the large crystals of cinacalcet hydrochloride of the invention have a diameter less than 550 μm.

In a further aspect, the large crystals of cinacalcet hydrochloride with a narrow particle size distribution obtained by the process of the invention have a particle size distribution in which approximately 50% of the total volume comprises particles having a diameter of approximately 100 μm or below and approximately 90% of the total volume comprises particles having a diameter of approximately 550 μm or below.

Another aspect of the invention relates to the use of the large crystals of cinacalcet hydrochloride obtained according to the process of the invention, wherein said large crystals show a narrow particle size distribution, to prepare small crystals of cinacalcet hydrochloride with a narrow particle size distribution. Thus, another aspect of the invention relates to a process for preparing small crystals of cinacalcet hydrochloride with a narrow particle size distribution, said process comprising reducing the particle size of the large crystals of cinacalcet hydrochloride of the invention by means of a conventional mechanical process of reducing the size of particles.

The reduction of particle size may be achieved via any conventional mechanical process of reducing the size of particles which includes any one or more of cutting, chipping, grinding, crushing, milling, micronizing, and trituration.

In one embodiment of the invention, the reducing the particle size of the large crystals of cinacalcet hydrochloride of the invention is carried out by means of a milling process which comprises rapid vibration of three spheres inside a capsule containing a sample of said large crystals of cinacalcet hydrochloride. More precisely, the milling is carried out in a Specac Specamill apparatus adjusted to maximum amplitude of vibration, using three agate balls as spheres, an agate capsule, and for 1 hour.

In another aspect, the small crystals of cinacalcet hydrochloride with a narrow particle size distribution obtained by the process of the invention show a D₅₀ equal or less than 50 μm. In certain embodiments, the small crystals of cinacalcet hydrochloride obtained by the process of the invention have a distribution of D_{[v, 0.5]} of 21.2 μm to 25.7 μm.
Another aspect of the invention includes a pharmaceutical composition including cinacalcet hydrochloride obtained according to the processes of the invention.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

The following examples are for illustrative purposes only and are not intended, nor should they be interpreted, to limit the scope of the invention.

### SPECIFIC EXAMPLES

### General Experimental Conditions

1. Particle Size Distribution Method

The particle size for cinacalcet hydrochloride was measured using a Malvern Mastersizer S particle size analyzer with an MS1 Small Volume Sample Dispersion Unit stirred cell. A 300RF mm lens and a beam length of 2.4 mm were used. Samples for analysis were prepared by dispersing a weighed amount of cinacalcet hydrochloride (approximately 60 mg) in 20 mL of sample dispersant, previously prepared by dilution of 1.5 g of Soybean Lecithin to 200 mL with Isopar G. The suspension was delivered drop-wise to the background-corrected measuring cell filled with dispersant (Isopar G) until the obscuration reached the desired level. Nine repeated readings of the volume distributions were taken. For characterization, the values of D₅₀ and D₉₀ (by volume) were selected and reported as the mean of the nine values measured.

### Example 1

### Crystallization of Cinacalcet Hydrochloride Under Uncontrolled Cooling Conditions

20.15 g of cinacalcet hydrochloride and 141 mL of isobutyl acetate were loaded into a 250 mL glass round-bottomed glass reactor and heated until dissolution occurred (at about 106° C.). The solution was then cooled down to 5° C. over 45 min at a stir speed of about 240 rpm (i.e. the mean cooling rate was about 135° C./h). The suspension was stirred at this temperature for a further hour and then filtered. The collected solid was washed with 18 mL of isobutyl acetate and dried under vacuum for 4 h at 60° C. This yielded 93% of cinacalcet hydrochloride with a particle size of D_{[v, 0.5]}: 23.4 μm; D_{[v, 0.9]}: 95.4 μm.

### Example 2

### Crystallization of Cinacalcet Hydrochloride Under Uncontrolled Cooling Conditions

20.00 g of cinacalcet hydrochloride and 140 mL of isobutyl acetate were loaded into a 500 mL round-bottomed glass reactor and heated until dissolution occurred (at about 109° C.). The solution was then cooled down to 83° C. at a stir speed of about 400 rpm, at which point 50 mL of n-heptane were added. The resulting suspension was further cooled down to 5° C. while stirring at about 400 rpm. The total cooling time was about 75 min (i.e. the mean cooling rate was about 80° C./h). The suspension was stirred at this temperature for a further hour and then filtered. The collected solid was washed with 17 mL of isobutyl acetate and dried under vacuum for 4 h at 60° C. This yielded 89% of cinacalcet hydrochloride with a particle size of D_{[v, 0.5]}: 20.7 μm; D_{[v, 0.9]}: 86.1 μm.

### Example 3

### Crystallization of Cinacalcet Hydrochloride Under Uncontrolled Cooling Conditions

20.07 g of cinacalcet hydrochloride and 140 mL of isobutyl acetate were loaded into a 500 mL round-bottomed glass reactor and heated until dissolution occurred (at about 107° C.). The solution was then cooled down to 85° C. over 1 h at a stir speed of about 60 rpm (i.e. the mean cooling rate was about 22° C./h). After this period, 50 mL of n-heptane were added to the stirred suspension, which was then cooled down to 5° C. over a further 3 hours. The suspension was stirred at this temperature for a further hour and then filtered. The collected solid was washed with 17 mL of isobutyl acetate and dried under vacuum for 4 h at 60° C. This yielded 89% of cinacalcet hydrochloride with a particle size of D_{[v, 0.5]}: 49.8 μm; D_{[v, 0.9]}: 387.9 μm.

### Example 4

### Crystallization of Cinacalcet Hydrochloride Under Controlled Cooling Conditions

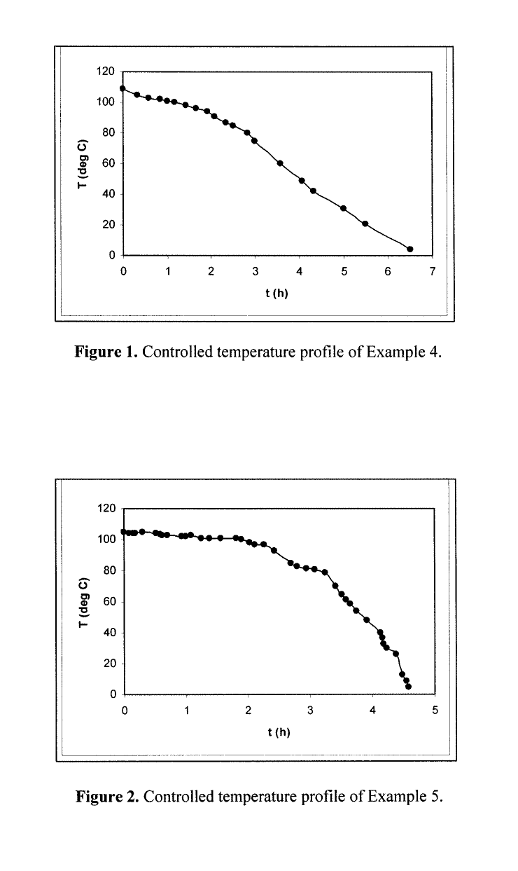

20.01 g of cinacalcet hydrochloride and 140 mL of isobutyl acetate were loaded into a 500 mL round-bottomed glass reactor and heated until dissolution occurred (at about 109° C.). Seeding with 0.05% w/w 50 μm (D₅₀) cinacalcet hydrochloride was performed at 105° C. The mixture was then cooled down to 5° C. over a total period of 6.5 h, by following the controlled temperature profile shown in FIG. 1 (i.e. the mean cooling rate in the region 105-75° C. was about 11° C./h). The stir speed was about 60 rpm. The suspension was stirred at 5° C. for an additional 1 h and then filtered. The collected solid was dried under vacuum for 4 h at 60° C. This yielded 90% of cinacalcet hydrochloride with a particle size of D_{[v, 0.5]}: 57.7 μm; D_{[v, 0.9]}: 403.9 μM.

### Example 5

### Crystallization of Cinacalcet Hydrochloride Under Controlled Cooling Conditions

26.36 g of cinacalcet hydrochloride and 185 mL of isobutyl acetate were loaded into a 250 mL round-bottomed glass reactor and heated to reflux (i.e. 118° C.) until dissolution occurred. Seeding with 1.0% w/w 38 μm (D₅₀) cinacalcet hydrochloride was performed at 105° C. The mixture was then cooled down to 5° C. over a total period of 4.5 h, by following the temperature profile shown in FIG. 2 (i.e. the mean cooling rate in the region 105-75° C. was about 9° C./h). The suspension was stirred at 5° C. for an additional 1 h and then filtered. The collected solid was washed with 25 mL of isobutyl acetate and then dried under vacuum for 4 h at 60° C. This yielded 99% of cinacalcet hydrochloride with a particle size of D_{[v, 0.5]}: 76.4 μm; D_{[v, 0.9]}: 456.0 μm.

### Example 6

### Crystallization of Cinacalcet Hydrochloride Under Controlled Cooling Conditions

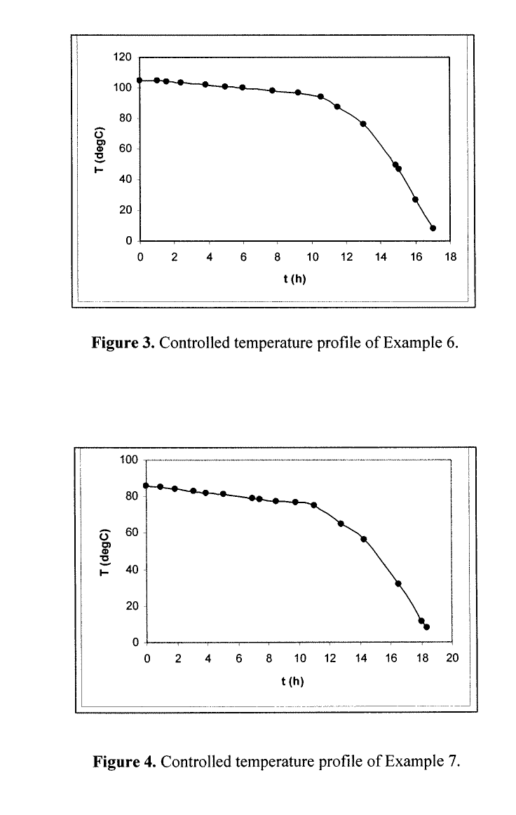

220.30 g of cinacalcet hydrochloride and 1540 mL of isobutyl acetate were loaded into a 2 L jacketed glass reactor and heated to reflux until dissolution occurred. Seeding with 0.5% w/w 38 μm (D₅₀) cinacalcet hydrochloride was performed at 105° C. The mixture was cooled down to 5° C. over a total period of 17 h, by following the temperature profile shown in FIG. 3 (i.e. the mean cooling rate in the region 105-90° C. was about 1° C./h). The stir speed was about 60 rpm. The suspension was stirred at 5° C. for an additional hour and then filtered. The collected solid was washed with 220 mL of isobutyl acetate and dried under vacuum for 4 h at 60° C. This yielded 96% of cinacalcet hydrochloride with a particle size of D_{[v, 0.5]}: 65.0 μm; D_{[v, 0.9]}: 392.3 μm.

### Example 7

### Crystallization of Cinacalcet Hydrochloride Under Controlled Cooling Conditions

193.36 g of cinacalcet hydrochloride, 1354 mL of isobutyl acetate and 12 mL of water were loaded into a 2 L jacketed glass reactor and heated to reflux until dissolution occurred. The solution was cooled down to 88° C. At this temperature crystallization was observed. The mixture was then cooled down to 5° C. over a total period of 19 h, by following the temperature profile shown in FIG. 4 (i.e. the mean cooling rate in the region 88-75° C. was about 1° C./h). The stir speed was about 100 rpm. The suspension was stirred at 5° C. for an additional hour and then filtered. The collected solid was washed with 200 mL of isobutyl acetate and then dried under vacuum for 4 h at 60° C. This yielded 95% of cinacalcet hydrochloride with a particle size of D_{[v, 0.5]}: 70.3 μm; D_{[v, 0.9]}: 431.7 μm.

### Example 8

### Crystallization of Cinacalcet Hydrochloride Under Controlled Cooling Conditions

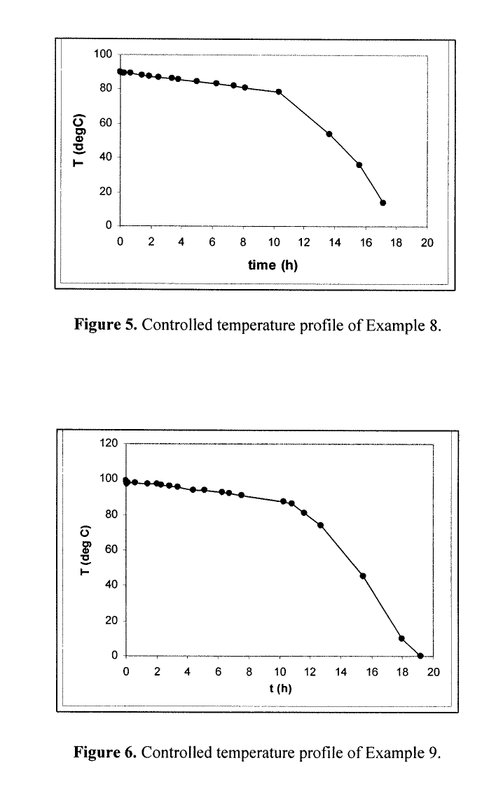

100.42 g of cinacalcet hydrochloride and 1200 mL of isobutyl acetate were loaded into a 2 L jacketed glass reactor and heated to reflux until dissolution occurred. The solution was cooled down to 90° C. At this temperature crystallization was observed. The mixture was then cooled down to 5° C. over a total period of 17.5 h, by following the temperature profile shown in FIG. 5 (i.e. the mean cooling rate in the region 90-75° C. was about 1° C./h). The suspension was stirred at 5° C. for an additional hour and then filtered. The collected solid was washed with 100 mL of isobutyl acetate and dried under vacuum for 4 h at 60° C. This yielded 96% of cinacalcet hydrochloride with a particle size of D_{[v, 0.5]}: 67.2 μm; D_{[v, 0.9]}: 459.0 μm.

### Example 9

### Crystallization of Cinacalcet Hydrochloride Under Controlled Cooling Conditions

90.10 g of cinacalcet hydrochloride and 1080 mL of isobutyl acetate were loaded into a 2 L jacketed glass reactor and heated to reflux until dissolution occurred. Seeding with 10% w/w 12.1 μm (D₅₀) cinacalcet hydrochloride (suspension in 25 mL isobutyl acetate) was performed at 99° C. The mixture was cooled down to 5° C. over a total period of 19 h, by following the temperature profile shown in FIG. 6 (i.e. the mean cooling rate in the region 99-85° C. was about 1° C./h). The suspension was stirred at 5° C. for an additional hour and then filtered. The collected solid was washed with 90 mL of isobutyl acetate and dried under vacuum for 4 h at 60° C. This yielded 94% of cinacalcet hydrochloride with a particle size of D_{[v, 0.5]}: 74.0 μm; D_{[v, 0.9]}: 518.3 μn.

### Example 10

### Preparation of Small Crystals of Cinacalcet Hydrochloride

Samples of the cinacalcet hydrochloride obtained from Examples 4, 7 and 9 were milled as follows:

A 200 mg sample of cinacalcet hydrochloride was introduced to an agate capsule with three agate balls. The closed capsule was mounted on a Specac Specamill apparatus, adjusted to maximum amplitude of vibration, and milled for 1 hour.

The resultant products were analysed, and the following results were obtained:

TABLE 1

| | BEFORE MILLING | | AFTER MILLING | |
|---|---|---|---|---|
| Sample | D_{[v, 0 . . . 5]} | D_{[v, 0.9]} | D_{[v, 0 . . . 5]} | D_{[v, 0.9]} |
| Cinacalcet hydrochloride | 57.7 | 403.9 | 25.7 | 91.8 |
| from Example 4 | | | | |
| Cinacalcet hydrochloride | 70.3 | 431.7 | 21.2 | 83.3 |
| from Example 7 | | | | |
| Cinacalcet hydrochloride | 74.0 | 518.3 | 25.1 | 76.7 |
| from Example 9 | | | | |

### Example 11

### Crystallization of Cinacalcet Hydrochloride Under Controlled Cooling Conditions

When reproducing Example 8 under similar conditions at higher scale, the cinacalcet hydrochloride obtained had a particle size of D_{[v, 0.5]}: 99.8 μm; D_{[v, 0.9]}: 466.9 μm.

1.-8. (canceled)

9. Solid particles of crystalline cinacalcet hydrochloride, characterized by a D₅₀ higher than about 50 microns.

10. Solid particles of crystalline cinacalcet hydrochloride according to claim 9, further characterized by a narrow particle size distribution.

11. Solid particles of crystalline cinacalcet hydrochloride according to claim 9, further characterized by a D₅₀ in the range of about 50 to 100 microns.

12. Solid particles of crystalline cinacalcet hydrochloride according to claim 11, further characterized by a D₅₀ in the range of about 57.7 to 99.8 microns.

13. Solid particles of crystalline cinacalcet hydrochloride according to claim 9, further characterized by a D₉₀ lower than about 550 microns.

14. Solid particles of crystalline cinacalcet hydrochloride according to claim 9, further characterized by a D₉₀ in the range of about 392.3 to 518.3 microns.

15. A process of preparing solid particles of crystalline cinacalcet hydrochloride, characterized by a D₅₀ higher than about 50 microns, said process comprising crystallizing cinacalcet hydrochloride under controlled cooling conditions having a controlled mean cooling rate lower than about 22′C/hour.

16. A process according to claim 15, which comprises preparing solid particles of crystalline cinacalcet hydrochloride further characterized by a narrow particle size distribution.

17. A process according to claim 15, which comprises: (i) providing a hot solution of cinacalcet hydrochloride and a solvent comprising an organic solvent, wherein said hot solution has a temperature not less than about 75° C., (ii) allowing for the presence of crystals, at a temperature not less than about 75° C., (iii) cooling at a controlled mean cooling rate lower than about 22° C./hour until the temperature is reduced at least 10° C. to obtain a suspension, (iv) allowing the suspension to achieve at least room temperature, and (v) isolating solid particles of crystalline cinacalcet hydrochloride from said suspension.

18. A process according to claim 17, which further comprises drying the solid particles of crystalline cinacalcet hydrochloride isolated in step (v).

19. A process according to claim 17, wherein the solvent comprising an organic solvent is at least one organic solvent or a mixture of at least one organic solvent and water.

20. A process according to claim 19, wherein the solvent comprising an organic solvent is isobutyl acetate, or a mixture of isobutyl acetate and water.

21. A process according to claim 17, wherein cooling at a controlled mean cooling rate lower than about 22° C./hour until the temperature is reduced at least 10° C. in step (iii) comprises cooling at a controlled mean cooling rate lower than about 22° C./hour until a temperature is reached within the range of about 85-65° C.

22. Solid particles of crystalline cinacalcet hydrochloride, obtained or obtainable by a process according to claim 15.

23. A process of preparing solid particles of crystalline cinacalcet hydrochloride characterized by a D₅₀ less than or equal to about 50 microns, which process comprises carrying out mechanical particle size reduction of solid particles of crystalline cinacalcet hydrochloride according to claim 9.

24. A process of preparing solid particles of crystalline cinacalcet hydrochloride characterized by a D₅₀ less than or equal to about 50 microns, which process comprises carrying out mechanical particle size reduction of solid particles of crystalline cinacalcet hydrochloride according to claim 22.

25. A solid pharmaceutical composition comprising solid particles of crystalline cinacalcet hydrochloride according to claim 9, and a pharmaceutically acceptable excipient.

26. A solid pharmaceutical composition comprising solid particles of crystalline cinacalcet hydrochloride according to claim 22, and a pharmaceutically acceptable excipient.

27. A method of treating secondary hyperparathyroidism in a subject afflicted with chronic kidney disease, which method comprises administering to the subject solid particles of crystalline cinacalcet hydrochloride according to claim 9.

28. A method of treating secondary hyperparathyroidism in a subject afflicted with chronic kidney disease, which method comprises administering to the subject solid particles of crystalline cinacalcet hydrochloride according to claim 22.

29. A method of treating hypercalcemia in a subject afflicted with parathyroid carcinoma, which method comprises administering to the subject solid particles of crystalline cinacalcet hydrochloride according to claim 9.

30. A method of treating hypercalcemia in a subject afflicted with parathyroid carcinoma, which method comprises administering to the subject solid particles of crystalline cinacalcet hydrochloride according to claim 22.
